(19) United States
(12) Patent Application Publication
(10) Pub. No.: US 2015/0126881 A1
(43) Pub. Date: May 7, 2015

(54) BIOLOGICAL INFORMATION MEASUREMENT DEVICE, PATIENT TERMINAL, SERVER, AND REMOTE REHABILITATION METHOD

(71) Applicants: Seiko Epson Corporation, Shinjuku-ku (JP); National University Corporation Nagoya University, Nagoya-shi (JP)

(72) Inventors: Takashi OGIUE, Shiojiri-shi (JP); Tadasuke Kotaki, Matsumoto-shi (JP); Sumio Yamada, Nagoya-shi (JP)

(21) Appl. No.: 14/594,024

(22) Filed: Jan. 9, 2015

Related U.S. Application Data
(63) Continuation of application No. PCT/JP2013/068818, filed on Jul. 10, 2013.

(30) Foreign Application Priority Data
Jul. 12, 2012 (JP) 2012-156247

(51) Int. Cl. A61B 5/0255; G06F 19/00; H04L 29/06; H04L 12/26; H04L 29/08

(52) U.S. Cl. CPC A61B 5/0255; H04L 43/045; H04L 67/10; H04L 67/42; G06F 19/3487
USPC 600/502; 709/203

(57) ABSTRACT

A biological information measurement device includes: a rehabilitation information reception section that receives rehabilitation information that includes information about a pulse rate range applied to a patient; a pulse rate range setting section that sets the pulse rate range based on the rehabilitation information; a display section that displays at least the pulse rate range; a pulse rate measurement section that measures a pulse rate of the patient; a storage section that stores pulse measurement information that includes the pulse rate measured by the pulse rate measurement section, and a measurement time at which the pulse rate was measured; and a pulse measurement information transmission section that transmits the pulse measurement information.

### CROSS REFERENCE TO RELATED APPLICATION

This application is a continuation of International Patent Application No. PCT/JP2013/068818, having an international filing date of Jul. 10, 2013, which designated the United States, the entirety of which is incorporated herein by reference. Japanese Patent Application No. 2012-156247 filed on Jul. 12, 2012 is also incorporated herein by reference in its entirety.

### BACKGROUND

The present invention relates to a biological information measurement device, a patient terminal, a server, a remote rehabilitation method, and the like.

In recent years, exercise rehabilitation that is designed to mainly improve lifestyle-related disease has been employed for patients with cardiac disease. When applying exercise rehabilitation to a patient with cardiac disease (i.e., a patient whose heart has been damaged), it is necessary to use a biological information measurement device that measures biological information (e.g., pulse rate) about the patient so that the patient can perform effective rehabilitation exercise with a moderate exercise stress while preventing a situation in which an undue stress is placed on the heart of the patient.

The number of patients who visit rehabilitation facilities has increased along with an increase in the number of patients with lifestyle-related disease (e.g., cardiac disease), and remote rehabilitation has been increasingly desired.

JP-A-2003-265441 discloses a biological activity measurement device that notifies the user that the pulse rate of the user has reached the pulse rate upper limit that is set in advance. JP-A-2003-265441 discloses measuring the effective cumulative time in which the pulse rate of the user was within the allowable pulse rate range, the upper-limit-exceeded cumulative time in which the pulse rate of the user was within the range of 10 beats above the upper-limit pulse rate, and the lower-limit-exceeded cumulative time in which the pulse rate of the user was within the range of 10 beats below the lower-limit pulse rate so that the user can determine the intensity of exercise and the exercise time corresponding to each intensity.

JP-A-2002-191718 discloses a remote rehabilitation system that is designed so that the rehabilitation instructor creates an exercise prescription program suitable for the patient, the exercise prescription program created by the rehabilitation instructor is transmitted to a patient-side terminal device through a communication line, and rehabilitation can be implemented according to the exercise prescription program using a rehabilitation device of the patient-side terminal device.

The biological activity measurement device disclosed in JP-A-2003-265441 is designed so that the pulse rate upper limit is set corresponding to the pulse rate prescribed by the doctor. However, since the patient must set the pulse rate upper limit through the liquid crystal display section, the biological activity measurement device disclosed in JP-A-2003-265441 is not necessarily convenient to the patient.

The remote rehabilitation system disclosed in JP-A-2002-191718 can transmit the exercise prescription program created by the rehabilitation instructor to the patient-side terminal device, and set the exercise prescription program to the patient-side terminal device. However, exercise indicated by the set exercise prescription program may not necessarily be optimum for the patient. For example, if a patient with cardiac disease performs exercise according to the set exercise prescription program when the physical condition of the patient is bad, an undue stress may be placed on the heart of the patient, and the life of the patient may be in danger.

### SUMMARY

According to one aspect of the invention, there is provided a biological information measurement device comprising:

a rehabilitation information reception section that receives rehabilitation information that includes information about a pulse rate range applied to a patient;

a pulse rate range setting section that sets the pulse rate range based on the rehabilitation information;

a display section that displays at least the pulse rate range;

a pulse rate measurement section that measures a pulse rate of the patient;

a storage section that stores pulse measurement information that includes the pulse rate measured by the pulse rate measurement section, and a measurement time at which the pulse rate was measured; and

a pulse measurement information transmission section that transmits the pulse measurement information.

According to another aspect of the invention, there is provided a patient terminal comprising:

a rehabilitation information acquisition section that acquires rehabilitation information from a server, the rehabilitation information including information about a pulse rate range applied to a patient;

a rehabilitation information transmission section that transmits the rehabilitation information to a biological information measurement device;

a pulse measurement information reception section that receives pulse measurement information from the biological information measurement device, the pulse measurement information including a measured pulse rate of the patient, and a measurement time at which the pulse rate was measured; and

a pulse measurement information transmission section that transmits the pulse measurement information to the server.

According to another aspect of the invention, there is provided a server comprising:

a rehabilitation information reception section that receives rehabilitation information from an instructor terminal, the rehabilitation information including information about a pulse rate range applied to a patient;

a rehabilitation information publication section that allows the rehabilitation information to be acquired from a patient terminal;

a pulse measurement information reception section that receives pulse measurement information from the patient terminal, the pulse measurement information including a measured pulse rate of the patient, and a measurement time at which the pulse rate was measured;

a document information generation section that generates document information based on the pulse measurement information, the document information being information about a pulse rate range measurement time in which the measured pulse rate was within the pulse rate range; and

a document information publication section that allows the document information to be viewed from the instructor terminal.

According to another aspect of the invention, there is provided a remote rehabilitation method that utilizes a server, an instructor terminal, a patient terminal, and a biological information measurement device, the remote rehabilitation method comprising:

causing the instructor terminal to transmit rehabilitation information to the server, the rehabilitation information including information about a pulse rate range applied to a patient;

causing the server to receive the rehabilitation information;

causing the server to allow the rehabilitation information to be acquired from the patient terminal;

causing the patient terminal to acquire the rehabilitation information from the server;

causing the patient terminal to transmit the rehabilitation information to the biological information measurement device;

causing the biological information measurement device to receive the rehabilitation information;

causing the biological information measurement device to set the pulse rate range based on the rehabilitation information;

causing the biological information measurement device to measure a pulse rate of the patient;

causing the biological information measurement device to store pulse measurement information that includes the measured pulse rate, and a measurement time at which the pulse rate was measured;

causing the biological information measurement device to transmit the pulse measurement information to the patient terminal;

causing the patient terminal to receive the pulse measurement information;

causing the patient terminal to transmit the pulse measurement information to the server;

causing the server to receive the pulse measurement information;

causing the server to generate document information based on the pulse measurement information, the document information being information about a pulse rate range measurement time in which the measured pulse rate was within the pulse rate range; and

causing the server to allow the document information to be viewed from the instructor terminal.

### BRIEF DESCRIPTION OF THE DRAWINGS

FIG. 1 is a view illustrating the configuration of a remote rehabilitation system according to a first embodiment.

FIG. 2 is a functional block diagram of an instructor terminal.

FIG. 3 is a view illustrating the data structure of rehabilitation information.

FIG. 4 is a functional block diagram of a server.

FIG. 5 is a view illustrating the data structure of exercise data.

FIG. 6 is a view illustrating an exercise report generated by a server.

FIG. 7 is a functional block diagram of a patient terminal.

FIG. 8 is a view illustrating the external configuration of a biological information measurement device according to the first embodiment.

FIG. 9 is a functional block diagram of a biological information measurement device.

FIG. 10 is a view illustrating a screen displayed on a biological information measurement device.

FIG. 11 is a flowchart illustrating a remote rehabilitation method.

FIG. 12 is a functional block diagram of a biological information measurement device according to a second embodiment.

FIG. 13 is a view illustrating an example of a screen displayed on a biological information measurement device according to the second embodiment.

FIG. 14 is a view illustrating an example of a screen displayed on a biological information measurement device according to a third embodiment.

FIG. 15 is a view illustrating an exercise report generated by a server according to the third embodiment.

FIG. 16 is a view illustrating a screen displayed on a biological information measurement device according to a modification.

FIG. 17 is a view illustrating a screen displayed on a biological information measurement device according to a modification.

### DESCRIPTION OF EXEMPLARY EMBODIMENTS

Several embodiments of the invention may implement a biological information measurement device, a patient terminal, a server, and a remote rehabilitation method that are convenient for the patient, and can implement rehabilitation exercise corresponding to the physical condition of the patient.

Several embodiments of the invention may be implemented by the following application examples.

### Application Example 1

A biological information measurement device according to Application Example 1 comprising:

a rehabilitation information reception section that receives rehabilitation information that includes information about a pulse rate range applied to a patient;

a pulse rate range setting section that sets the pulse rate range based on the rehabilitation information;

a display section that displays at least the pulse rate range;

a pulse rate measurement section that measures a pulse rate of the patient;

a storage section that stores pulse measurement information that includes the pulse rate measured by the pulse rate measurement section, and a measurement time at which the pulse rate was measured; and

a pulse measurement information transmission section that transmits the pulse measurement information.

According to Application Example 1, the biological information measurement device can receive the rehabilitation information including the information about the pulse rate range applied to the patient from the outside, and set the pulse rate range applied to the biological information measurement device based on the received rehabilitation information. This makes it unnecessary for the patient to set the pulse rate range, and makes it possible to improve convenience to the patient. Since the instructor (e.g., doctor) can remotely set the pulse rate range applied to the patient, the instructor can set the pulse rate range suitable for the patient at an appropriate timing corresponding to an improvement in the health condition of the patient through rehabilitation. Therefore, the effects of remote rehabilitation can be further improved.

According to Application Example 1, the biological information measurement device receives the rehabilitation information including the information about the pulse rate range from the outside instead of receiving an exercise prescription program. This makes it possible to implement rehabilitation exercise corresponding to the physical condition of the patient. When the physical condition of the patient is bad, the pulse rate of the patient normally easily reaches the set pulse rate range even when the patient performs relatively mild exercise. When the physical condition of the patient is good, the pulse rate of the patient does not reach the set pulse rate range unless the patient performs relatively hard exercise. Specifically, it is possible to implement safe rehabilitation exercise corresponding to the physical condition of the patient by setting the pulse rate range.

For example, the pulse rate of the patient during exercise is measured, the pulse measurement information that includes the measured pulse rate and the measurement time at which the pulse rate was measured is stored, and the stored pulse measurement information is transmitted to the outside. This makes it possible to remotely report the status of implementation of rehabilitation exercise performed by the patient to the instructor, and the instructor can determine an improvement in the health condition of the patient based on the status of implementation of rehabilitation exercise performed by the patient. It is also possible to set a new pulse rate range suitable for the patient at an appropriate timing corresponding to an improvement in the health condition of the patient, for example.

The display section displays at least the pulse rate range. This makes it possible to allow the patient to be aware of the pulse rate range set for the patient, and easily implement effective rehabilitation exercise. The inventors of the invention conducted extensive studies, and found that the effects of rehabilitation exercise performed by the patient are improved as the time in which the patient exercised within the pulse rate range set by the instructor (e.g., doctor) increases. It is possible to allow the patient to be aware of the pulse rate range set for the patient, and prompt the patient to exercise within the set pulse rate range by displaying the set pulse rate range.

### Application Example 2

The biological information measurement device according to Application Example 1 may further comprise:

a pulse rate range measurement time calculation section that calculates a pulse rate range measurement time in which the measured pulse rate was within the pulse rate range,

the display section may display the pulse rate range measurement time.

According to Application Example 2, the biological information measurement device calculates the pulse rate range measurement time in which the measured pulse rate was within the pulse rate range, and displays the calculated pulse rate range measurement time. This makes it possible to prompt the patient to exercise so that the pulse rate range measurement time increases, and allow the patient to determine whether or not the patient could perform effective rehabilitation exercise.

### Application Example 3

The biological information measurement device according to Application Example 2,

the pulse rate range measurement time calculation section may calculate an upper range measurement time in which the measured pulse rate was within a given range from an upper limit of the pulse rate range, and

the display section may display at least the upper range measurement time.

According to Application Example 3, the biological information measurement device calculates the upper range measurement time in which the measured pulse rate was within a given range from the upper limit of the pulse rate range, and displays the upper range measurement time. The inventors found as a result of extensive studies that the effects of rehabilitation exercise performed by the patient are improved when the patient exercises for a long time at a pulse rate within a range close to the upper limit of the pulse rate range. Therefore, it is possible to allow the patient to determine whether or not the patient could perform effective rehabilitation exercise by displaying the upper range measurement time in which the pulse rate of the patient was within a given range from the upper limit of the pulse rate range.

### Application Example 4

The biological information measurement device according to Application Example 3 may further comprise:

a notification section that issues a notification to the patient when the pulse rate range measurement time has reached a first time.

According to Application Example 4, the biological information measurement device issues a notification to the patient when the pulse rate range measurement time has reached the first time. This makes it possible to notify the patient that the patient has performed sufficient rehabilitation exercise. Since the time in which the patient exercised within the pulse rate range contributes to an improvement in the effects of rehabilitation exercise performed by the patient, the biological information measurement device determines that the patient has performed sufficient rehabilitation exercise when the pulse rate range measurement time has reached the first time, and notifies the patient to that effect. This makes it possible to implement effective rehabilitation exercise.

### Application Example 5

The biological information measurement device according to Application Example 4 may further comprise:

an elapsed time measurement section that measures an elapsed time from start of the measurement of the pulse rate,

the notification section may issue a notification to the patient when the elapsed time has reached a second time that is longer than the first time.

According to Application Example 5, the biological information measurement device issues a notification to the patient when the elapsed time from the start of measurement of the pulse rate has reached the second time. The biological information measurement device determines that the patient has performed sufficient rehabilitation exercise when the pulse rate range measurement time has reached the first time, and notifies the patient to that effect. However, the biological information measurement device does not issue a notification to the patient when the pulse rate range measurement time has not reached the first time. In this case, it is difficult for the patient to determine whether or not the patient can stop exercise. When the patient is a patient with cardiac disease, excessive exercise may threaten the life of the patient. Therefore, the biological information measurement device notifies the patient that the patient can stop exercise when the elapsed time from the start of measurement of the pulse rate has reached the second time even when the pulse rate range measurement time has not reached the first time. This makes it possible to implement safe rehabilitation exercise.

### Application Example 6

A patient terminal in Application Example 6 may comprise:

a rehabilitation information acquisition section that acquires rehabilitation information from a server, the rehabilitation information including information about a pulse rate range applied to a patient;

a rehabilitation information transmission section that transmits the rehabilitation information to a biological information measurement device;

a pulse measurement information reception section that receives pulse measurement information from the biological information measurement device, the pulse measurement information including a measured pulse rate of the patient, and a measurement time at which the pulse rate was measured; and

a pulse measurement information transmission section that transmits the pulse measurement information to the server.

According to Application Example 6, the patient terminal acquires the rehabilitation information including the information about the pulse rate range applied to the patient from the server, and transmits the acquired rehabilitation information to the biological information measurement device. This makes it unnecessary for the patient to set the pulse rate range, and makes it possible to improve convenience to the patient. Since the instructor (e.g., doctor) can remotely set the pulse rate range applied to the patient, the instructor can set the pulse rate range suitable for the patient at an appropriate timing corresponding to an improvement in health condition of the patient through rehabilitation. Therefore, the effects of remote rehabilitation can be further improved. It is also possible to prevent a situation in which the patient erroneously sets the pulse rate range.

The patient terminal can receive the pulse measurement information including the measured pulse rate of the patient and the measurement time at which the pulse rate was measured, from the biological information measurement device, and transmit the pulse measurement information to the server. This makes it possible to remotely report the status of implementation of rehabilitation exercise performed by the patient to the instructor, and the instructor can determine an improvement in the health condition of the patient based on the status of implementation of rehabilitation exercise performed by the patient. It is also possible to set a new pulse rate range suitable for the patient at an appropriate timing corresponding to an improvement in the health condition of the patient, for example.

### Application Example 7

A server in Application Example 7 may comprise:

a rehabilitation information reception section that receives rehabilitation information from an instructor terminal, the rehabilitation information including information about a pulse rate range applied to a patient;

a rehabilitation information publication section that allows the rehabilitation information to be acquired from a patient terminal;

a pulse measurement information reception section that receives pulse measurement information from the patient terminal, the pulse measurement information including a measured pulse rate of the patient, and a measurement time at which the pulse rate was measured;

a document information generation section that generates document information based on the pulse measurement information, the document information being information about a pulse rate range measurement time in which the measured pulse rate was within the pulse rate range; and

a document information publication section that allows the document information to be viewed from the instructor terminal.

According to Application Example 7, the server receives the rehabilitation information including the information about the pulse rate range applied to the patient from the instructor terminal, and allows the received rehabilitation information to be acquired from the patient terminal. According to this configuration, since the instructor (e.g., doctor) can remotely set the pulse rate range applied to the patient, the instructor can set the pulse rate range suitable for the patient at an appropriate timing corresponding to an improvement in health condition of the patient through rehabilitation. Therefore, the effects of remote rehabilitation can be further improved. Since the patient need not set the pulse rate range by acquiring the rehabilitation information using the patient terminal, convenience to the patient is improved.

The server receives the pulse measurement information including the measured pulse rate of the patient and the measurement time at which the pulse rate was measured, from the patient terminal, generates the document information about the pulse rate range measurement time in which the measured pulse rate was within the pulse rate range based on the received pulse measurement information, and allows the generated document information to be viewed from the instructor terminal. This makes it possible to report the status of implementation of rehabilitation exercise performed by the patient to the instructor in a comprehensible manner, and the instructor can determine an improvement in the health condition of the patient based on the status of implementation of rehabilitation exercise performed by the patient. It is also possible to set a new pulse rate range suitable for the patient at an appropriate timing corresponding to an improvement in the health condition of the patient, for example.

### Application Example 8

A remote rehabilitation method in Application Example 8 that utilizes a server, an instructor terminal, a patient terminal, and a biological information measurement device, the remote rehabilitation method may comprise:

causing the instructor terminal to transmit rehabilitation information to the server, the rehabilitation information including information about a pulse rate range applied to a patient;

causing the server to receive the rehabilitation information;

causing the server to allow the rehabilitation information to be acquired from the patient terminal;

causing the patient terminal to acquire the rehabilitation information from the server;

causing the patient terminal to transmit the rehabilitation information to the biological information measurement device;

causing the biological information measurement device to receive the rehabilitation information;

causing the biological information measurement device to set the pulse rate range based on the rehabilitation information;

causing the biological information measurement device to measure a pulse rate of the patient;

causing the biological information measurement device to store pulse measurement information that includes the measured pulse rate, and a measurement time at which the pulse rate was measured;

causing the biological information measurement device to transmit the pulse measurement information to the patient terminal;

causing the patient terminal to receive the pulse measurement information;

causing the patient terminal to transmit the pulse measurement information to the server;

causing the server to receive the pulse measurement information;

causing the server to generate document information based on the pulse measurement information, the document information being information about a pulse rate range measurement time in which the measured pulse rate was within the pulse rate range; and

causing the server to allow the document information to be viewed from the instructor terminal.

According to Application Example 8, the instructor terminal can transmit the rehabilitation information including the information about the pulse rate range applied to the patient to the server to set the pulse rate range applied to the biological information measurement device through the server and the patient terminal. This makes it unnecessary for the patient to set the pulse rate range, and makes it possible to improve convenience to the patient. Since the instructor (e.g., doctor) can remotely set the pulse rate range applied to the patient, the instructor can set the pulse rate range suitable for the patient at an appropriate timing corresponding to an improvement in health condition of the patient through rehabilitation. Therefore, the effects of remote rehabilitation can be further improved.

According to Application Example 8, the instructor terminal transmits the rehabilitation information including the information about the pulse rate range instead of transmitting an exercise prescription program. This makes it possible to implement safe rehabilitation exercise corresponding to the physical condition of the patient, as described above in connection with Application Example 1.

The server receives the pulse measurement information including the pulse rate measured by the biological information measurement device and the measurement time at which the pulse rate was measured, through the patient terminal, generates the document information about the pulse rate range measurement time in which the measured pulse rate was within the pulse rate range based on the received pulse measurement information, and allows the generated document information to be viewed from the instructor terminal. This makes it possible to report the status of implementation of rehabilitation exercise performed by the patient to the instructor in a comprehensible manner, and the instructor can determine an improvement in the health condition of the patient based on the status of implementation of rehabilitation exercise performed by the patient. It is also possible to set a new pulse rate range suitable for the patient at an appropriate timing corresponding to an improvement in the health condition of the patient, for example.

The embodiments of the invention are described below in the following order so that the invention can be clearly and sufficiently understood.

1. First embodiment

1-1. Configuration of remote rehabilitation system
1-1-1. Configuration of instructor terminal
1-1-2. Configuration of server
1-1-3. Configuration of patient terminal
1-1-4. Configuration of biological information measurement device

1-2. Process of remote rehabilitation method

1-3. Advantageous effects

2. Second embodiment
3. Third embodiment

### 4. Modifications

### 1. First Embodiment

### 1-1. Configuration of Remote Rehabilitation System

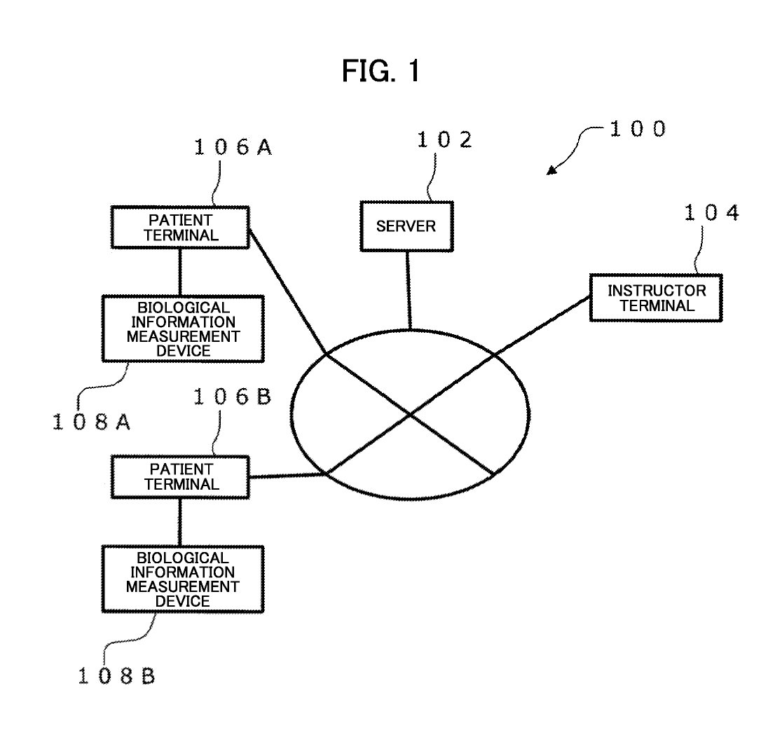

FIG. 1 is a system configuration diagram illustrating a remote rehabilitation system 100 that implements a remote rehabilitation method according to the first embodiment. The first embodiment illustrates an example of remote rehabilitation for a patient with heart disease (e.g., myocardial infarction or angina pectoris). A remote rehabilitation system 100 includes an instructor terminal 104, a server 102, a patient terminal 106 (106A, 106B, . . . ), and a biological information measurement device 108 (108A, 108B, . . . ). The server 102, the instructor terminal 104, and the patient terminal 106 are connected through a network, and exchange various types of information.

The instructor terminal 104 is a terminal that is used by an instructor (e.g., doctor) who issues instructions relating to remote rehabilitation. The patient terminal 106 can be connected to the biological information measurement device 108, and can exchange various types of information with the biological information measurement device 108. The biological information measurement device 108 is attached to the body of the patient, and measures biological information about the patient. Examples of the biological information include the body temperature, the blood pressure, the pulse rate, and the like of the patient. Note that the first embodiment illustrates an example in which the biological information is the pulse rate.

Although FIG. 1 illustrates an example in which two patient terminals 106 and two biological information measurement devices 108 are provided, the configuration is not limited thereto. Specifically, the remote rehabilitation system 100 includes the patient terminal 106 and the biological information measurement device 108 corresponding to each patient. Each patient terminal 106 and each biological information measurement device 108 respectively have an identical configuration. The following description is given taking one patient terminal 106 and one biological information measurement device 108 for convenience of explanation.

### 1-1-1. Configuration of Instructor Terminal

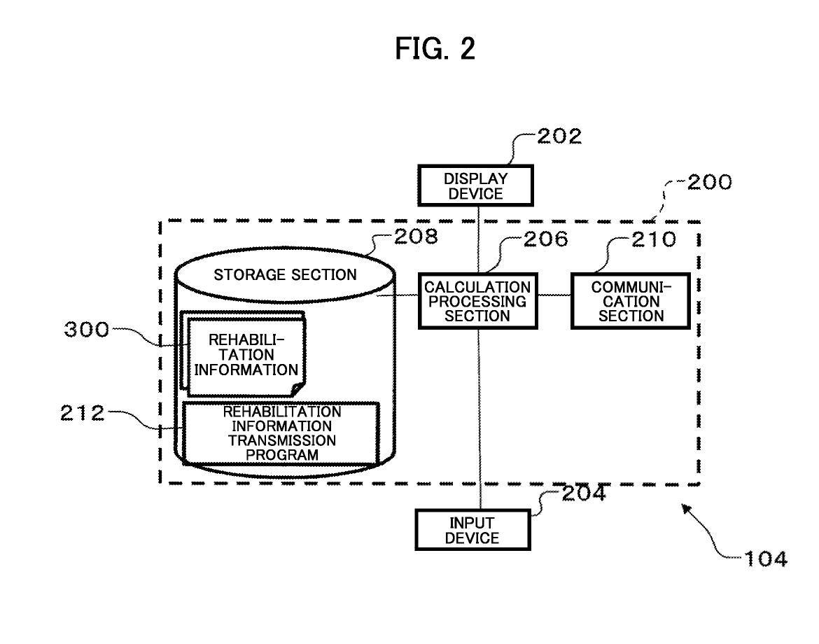

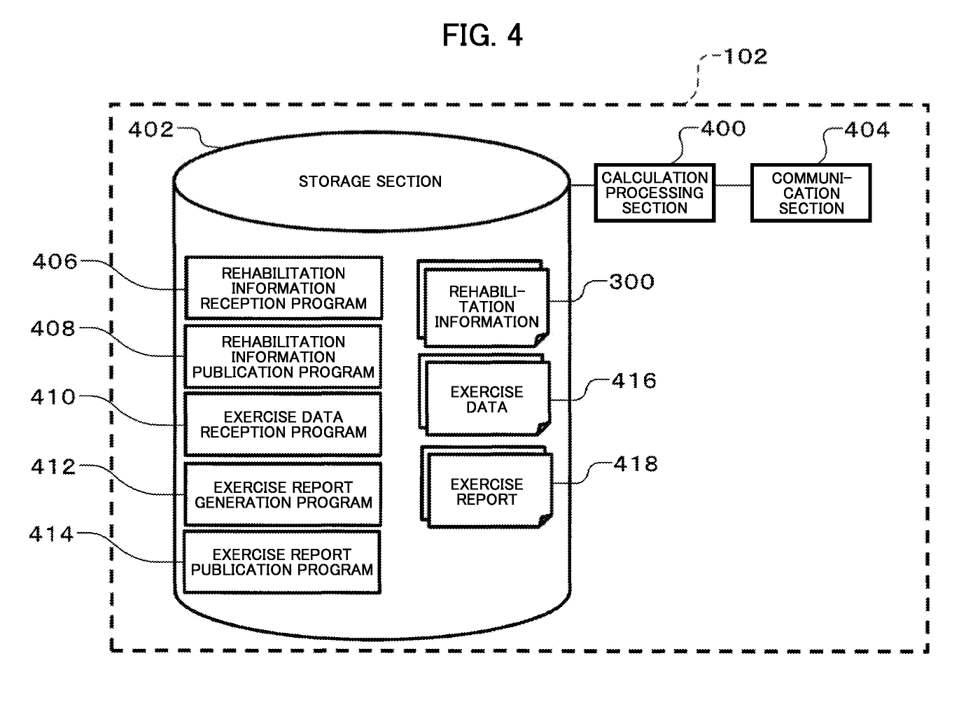

FIG. 2 is a functional block diagram of the instructor terminal 104. The instructor terminal 104 is a personal computer (PC). The instructor terminal 104 transmits rehabilitation information 300 (described later) to the server 102, and allows the instructor to view an exercise report 418 (see FIG. 4) provided by the server 102. The instructor terminal 104 includes a computer main body 200, a display device 202, and an input device 204. The computer main body 200 includes a calculation processing section 206, a communication section 210, and a storage section 208.

The storage section 208 stores data and a program. Examples of the data stored in the storage section 208 include the rehabilitation information 300 about rehabilitation that is suitable for the patient. The rehabilitation information 300 is stored corresponding to each patient. Examples of the program stored in the storage section 208 include a rehabilitation information transmission program 212 for transmitting the rehabilitation information 300 to the server 102.

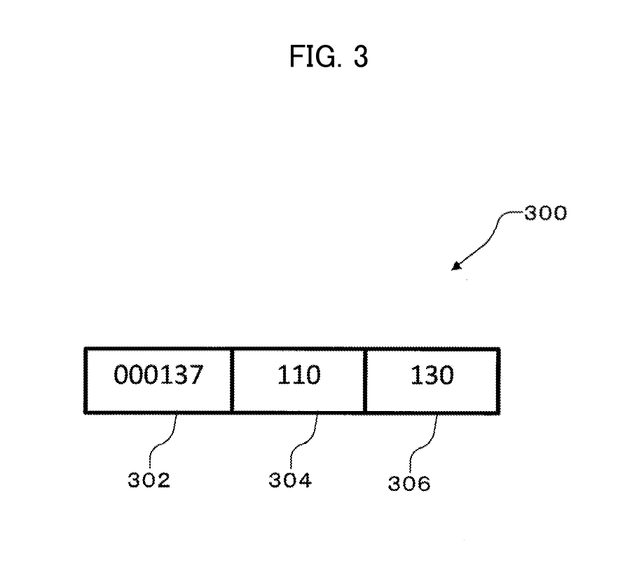

FIG. 3 is a view illustrating the data structure of the rehabilitation information 300. The rehabilitation information 300 includes a patient ID 302 that is identification information about the patient, a lower-limit pulse rate 304 that represents the lower limit of a pulse rate range applied to the patient, and an upper-limit pulse rate 306 that represents the upper limit of the pulse rate range. FIG. 3 illustrates an example in which “000137” is stored in the storage section 208 as the patient ID 302, “110” is stored in the storage section 208 as the lower-limit pulse rate 304, and “130” is stored in the storage section 208 as the upper-limit pulse rate 306. The rehabilitation information 300 is transmitted to the server 102 when the rehabilitation information transmission program 212 is executed.

The communication section 210 is a network connection interface.

The calculation processing section 206 controls each section and each device included in the instructor terminal 104. The calculation processing section 206 reads a program stored in the storage section 208, and executes the program. The calculation processing section 206 is a central processing unit (CPU), for example. The calculation processing section 206 reads the rehabilitation information transmission program 212 from the storage section 208, and executes the rehabilitation information transmission program 212 to transmit the rehabilitation information 300 to the server 102 through the communication section 210. Specifically, the calculation processing section 206 functions as a rehabilitation information transmission section.

The display device 202 displays the results of calculations performed by the calculation processing section 206, and displays a screen interface for receiving an input from the input device 204. The display device 202 is a liquid crystal display, for example.

The input device 204 is a user interface that is used when inputting instructions and data to the calculation processing section 206. The input device 204 may be implemented by a keyboard or a mouse, for example. The input device 204 may be implemented by a touch panel display in which the input device 204 is integrated with the display device 202.

### 1-1-2. Configuration of Server

FIG. 4 is a functional block diagram of the server 102. The server 102 is a network server. The server 102 preferably has a World Wide Web (WWW) server function. The server 102 includes a calculation processing section 400, a storage section 402, and a communication section 404.

The storage section 402 stores data and a program. Examples of the data stored in the storage section 402 include the rehabilitation information 300 received from the instructor terminal 104, exercise data 416 received from the patient terminal 106, and the exercise report 418 that is generated by the server 102 using the exercise data 416. The rehabilitation information 300, the exercise data 416, and the exercise report 418 are stored corresponding to each patient. Examples of the program stored in the storage section 402 include a rehabilitation information reception program 406 for receiving the rehabilitation information 300 from the instructor terminal 104, a rehabilitation information publication program 408 for allowing the patient terminal 106 to acquire the rehabilitation information 300, an exercise data reception program 410 for receiving the exercise data 416 from the patient terminal 106, an exercise report generation program 412 for generating the exercise report 418, and an exercise report publication program 414 for allowing the exercise report 418 to be viewed from the instructor terminal 104.

The communication section 404 is a network connection interface.

The calculation processing section 400 controls each section included in the server 102. The calculation processing section 400 reads a program stored in the storage section 402, and executes the program. The calculation processing section 400 is a CPU, for example.

The calculation processing section 400 reads the rehabilitation information reception program 406 from the storage section 402, and executes the rehabilitation information reception program 406 to receive the rehabilitation information 300 from the instructor terminal 104 through the communication section 404. Specifically, the calculation processing section 400 functions as a rehabilitation information reception section.

The calculation processing section 400 reads the rehabilitation information publication program 408 from the storage section 402, and executes the rehabilitation information publication program 408 to allow the patient terminal 106 to acquire the rehabilitation information 300 (i.e., store the rehabilitation information 300 in a given folder that is open to the public as a WWW server). Specifically, the calculation processing section 400 functions as a rehabilitation information publication section.

The calculation processing section 400 reads the exercise data reception program 410 from the storage section 402, and executes the exercise data reception program 410 to receive the exercise data 416 from the patient terminal 106 through the communication section 404. Specifically, the calculation processing section 400 functions as an exercise data reception section (also referred to as “pulse measurement information reception section”).

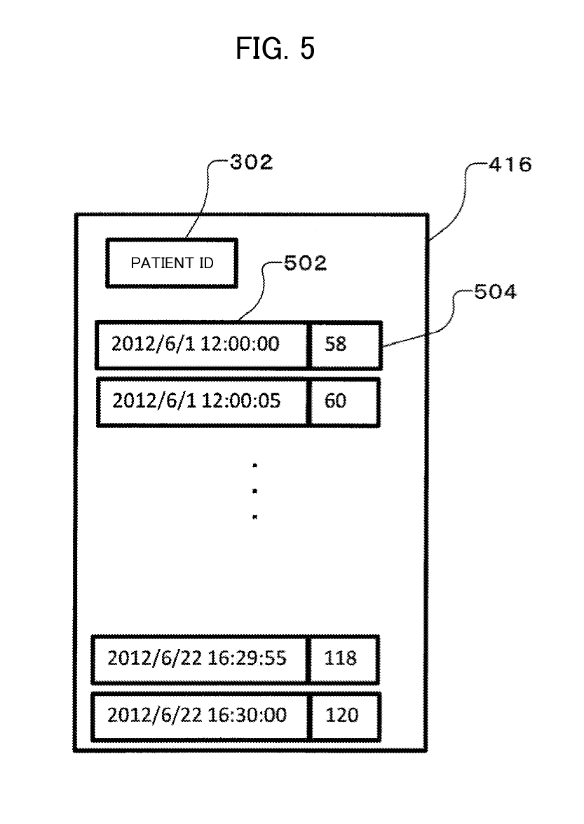

FIG. 5 is a view illustrating the data structure of the exercise data 416. The exercise data 416 is data relating to the pulse rate of the patient measured by the biological information measurement device 108 (described later). The exercise data 416 includes a patient ID 302, measurement date/time data 502, and pulse rate data 504. Note that the exercise data 416 is also referred to as “pulse measurement information”.

The patient ID 302 is identification information about the patient, and is linked to the patient ID 302 included in the rehabilitation information 300. Specifically, the patient ID 302 included in the rehabilitation information 300 and the patient ID 302 included in the exercise data 416 are identical on a patient basis.

The measurement date/time data 502 and the pulse rate data 504 are linked to each other. The pulse rate data 504 represents the pulse rate measured at the date/time represented by the measurement date/time data 502. The exercise data 416 includes the measurement date/time data 502 and the pulse rate data 504 during a given period. In the example illustrated in FIG. 5, the exercise data 416 includes the measurement date/time data 502 and the pulse rate data 504 during a three-week period. Note that the configuration is not limited thereto. For example, the exercise data 416 may include the measurement date/time data 502 and the pulse rate data 504 during a one-day period, or may include the measurement date/time data 502 and the pulse rate data 504 during a one-month period.

Again referring to FIG. 4, the calculation processing section 400 reads the exercise report generation program 412 from the storage section 402, and executes the exercise report generation program 412 to generate the exercise report 418 from the exercise data 416 stored in the storage section 402. Specifically, the calculation processing section 400 functions as an exercise report generation section (also referred to as “document information generation section”).

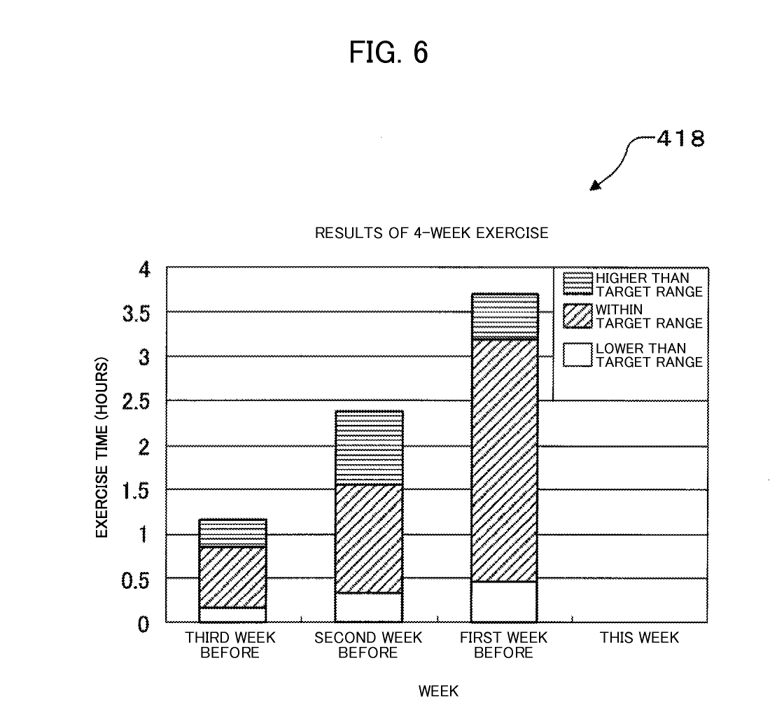

FIG. 6 is a view illustrating an example of the exercise report 418 generated by the exercise report generation program 412. The exercise report generation program 412 generates the exercise report 418 corresponding to each patient referring to the exercise data 416 and the rehabilitation information 300 stored in the storage section 402. The exercise report 418 is preferably generated in a Hyper Text Markup Language (HTML) format. Note that the exercise report 418 is also referred to as “document information”.

In FIG. 6, the horizontal axis represents that four weeks are divided on a weekly basis, and the vertical axis represents the cumulative exercise time (hours) during a one-week period. In the first embodiment, the pulse is measured when the patient exercises. Therefore, the pulse measurement time is considered to be the exercise time of the patient.

Each vertical bar graph illustrated in FIG. 6 represents the total exercise time during a one-week period. The white part of each vertical bar graph represents the cumulative time in which the patient exercised at a pulse rate lower than a pulse rate range (target) suitable for the patient. The diagonally shaded part of each vertical bar graph represents the cumulative time in which the patient exercised at a pulse rate within the pulse rate range suitable for the patient. The horizontally shaded part of each vertical bar graph represents the cumulative time in which the patient exercised at a pulse rate higher than the pulse rate range suitable for the patient.

Again referring to FIG. 4, the calculation processing section 400 reads the exercise report publication program 414 from the storage section 402, and executes the exercise report publication program 414 to allow the exercise report 418 stored in the storage section 402 to be viewed from the instructor terminal 104 (i.e., store the exercise report 418 in a given folder that is open to the public as a WWW server). Specifically, the calculation processing section 400 functions as an exercise report publication section (also referred to as “document information publication section”).

The instructor views the published exercise report 418 using the instructor terminal 104 to check and analyze the status of implementation of rehabilitation exercise by the patient.

### 1-1-3. Configuration of Patient Terminal

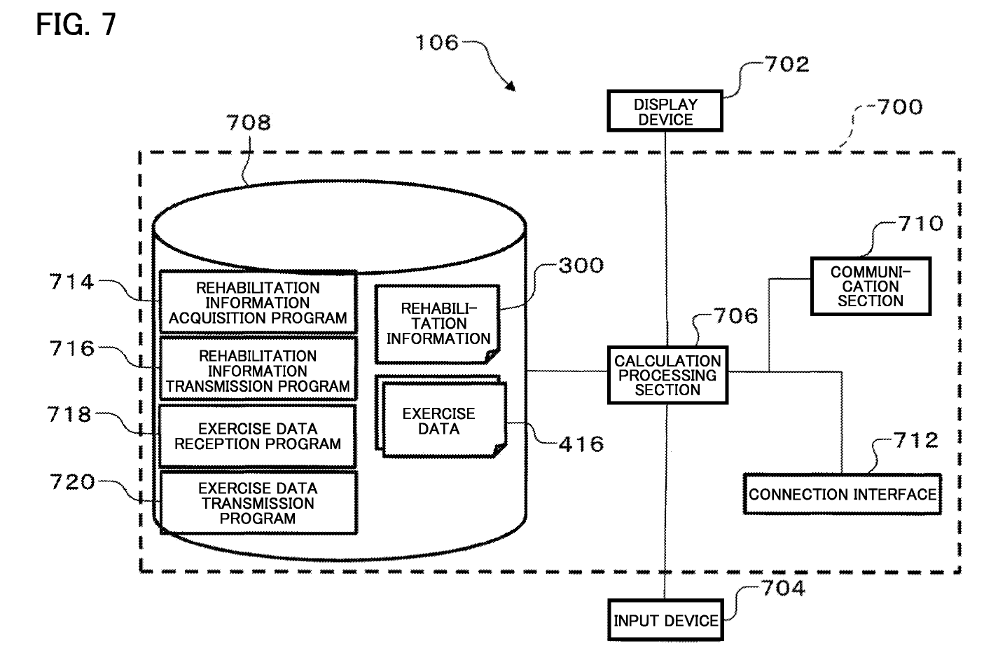

FIG. 7 is a functional block diagram of the patient terminal 106. The patient terminal 106 is a personal computer (PC). The patient terminal 106 includes a computer main body 700, a display device 702, and an input device 704. The computer main body 700 includes a calculation processing section 706, a communication section 710, a storage section 708, and a connection interface 712.

The storage section 708 stores data and a program. Examples of the data stored in the storage section 708 include the rehabilitation information 300 and the exercise data 416. Examples of the program stored in the storage section 708 include a rehabilitation information acquisition program 714 for acquiring the rehabilitation information 300 from the server 102, a rehabilitation information transmission program 716 for transmitting the rehabilitation information 300 to the biological information measurement device 108, an exercise data reception program 718 for receiving the exercise data 416 from the biological information measurement device 108, and an exercise data transmission program 720 for transmitting the exercise data 416 to the server 102.

The communication section 710 is a network connection interface.

The connection interface 712 is an interface for connecting to the biological information measurement device 108. The connection interface 712 is implemented by a serial interface (e.g., Universal Serial Bus (USB) or IEEE1394), for example.

The calculation processing section 706 controls each section and each device included in the patient terminal 106. The calculation processing section 706 reads a program stored in the storage section 708, and executes the program. The calculation processing section 706 is a CPU, for example.

The calculation processing section 706 reads the rehabilitation information acquisition program 714 from the storage section 708, and executes the rehabilitation information acquisition program 714 to acquire the rehabilitation information 300 from the server 102 through the communication section 710. Specifically, the calculation processing section 706 functions as a rehabilitation information acquisition section.

The calculation processing section 706 reads the rehabilitation information transmission program 716 from the storage section 708, and executes the rehabilitation information transmission program 716 to transmit the rehabilitation information 300 to the biological information measurement device 108 through the connection interface 712. Specifically, the calculation processing section 706 functions as a rehabilitation information transmission section.

The calculation processing section 706 reads the exercise data reception program 718 from the storage section 708, and executes the exercise data reception program 718 to receive the exercise data 416 from the biological information measurement device 108 through the connection interface 712. Specifically, the calculation processing section 706 functions as an exercise data reception section (also referred to as “pulse measurement information reception section”).

The calculation processing section 706 reads the exercise data transmission program 720 from the storage section 708, and executes the exercise data transmission program 720 to transmit the exercise data 416 from the server 102 through the communication section 710. Specifically, the calculation processing section 706 functions as an exercise data transmission section (also referred to as “pulse measurement information transmission section”).

The display device 702 displays the results of calculations performed by the calculation processing section 706, and displays a screen interface for receiving an input from the input device 704. The display device 702 is a liquid crystal display, for example.

The input device 704 is a user interface that is used when inputting instructions and data to the calculation processing section 706. The input device 704 may be implemented by a keyboard or a mouse, for example. The input device 704 may be implemented by a touch panel display in which the input device 704 is integrated with the display device 702.

### 1-1-4. Configuration of Biological Information Measurement Device

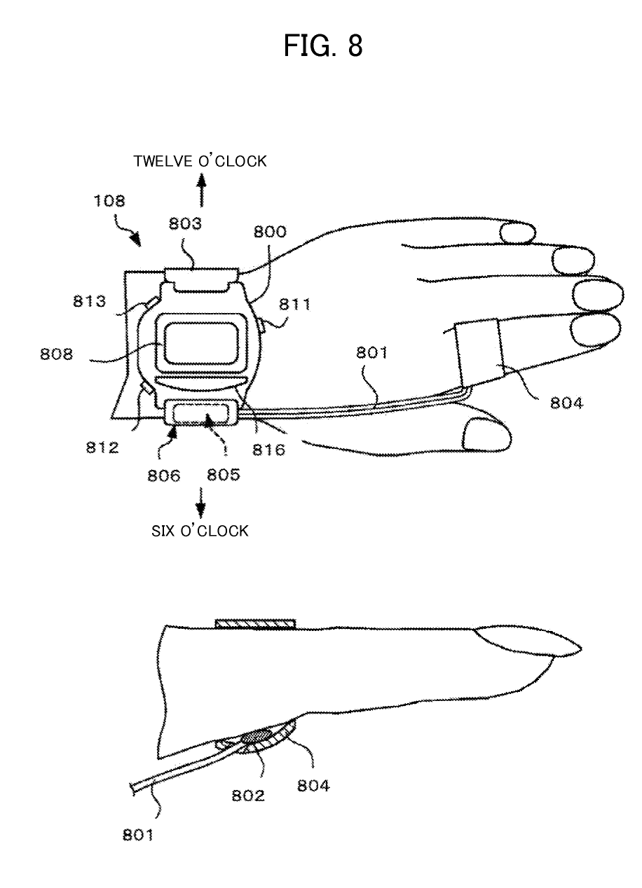

FIG. 8 is a view illustrating a schematic configuration of the wrist-worn biological information measurement device 108 according to the first embodiment, and the usage state of the biological information measurement device 108. The biological information measurement device 108 includes a main body 800 having a wristwatch-like structure. The main body 800 is provided with a wrist band 803 that is wound around the wrist of the patient (subject) from a position corresponding to the twelve o'clock direction, and secured at a position corresponding to the six o'clock direction. The main body 800 is designed to be removable from the wrist of the patient by adjusting the wrist band 803. In the first embodiment, the biological information measurement device 108 is a pulse rate meter.

The main body 800 includes a display section 808. The current time and the pulse rate of the patient are displayed on the display section 808 (described in detail later). A button switch 811 is provided to the periphery of the main body 800 at a position corresponding to the two o'clock direction. It is possible to change the image displayed on the display section 808 by pressing the button switch 811. A button switch 812 is provided to the periphery of the main body 800 at a position corresponding to the seven o'clock direction, and a button switch 813 is provided to the periphery of the main body 800 at a position corresponding to the eleven o'clock direction. The button switches 812 and 813 are used when the patient inputs information.

A start/stop button 816 is provided to the front side (i.e., the side where the display section 808 is provided) of the main body 800. The start/stop button 816 is used when the patient instructs the biological information measurement device 108 to start or stop pulse rate measurement during exercise.

A connector 805 is provided to the periphery of the main body 800 at a position corresponding to the six o'clock direction. A connector piece 806 is removably attached to the connector 805. One end of a cable 801 is connected to the connector piece 806. A pulse wave sensor unit 802 that measures the pulse rate of the patient is connected to the other end of the cable 801. The pulse wave sensor unit 802 is secured on the base of the finger of the patient using a sensor-securing band 804. Since the connector piece 806 is removably attached to the connector 805, the patient can use the biological information measurement device 108 as a wristwatch by removing the connector piece 806 from the connector 805.

The biological information measurement device 108 can be connected to the patient terminal 106 by attaching a cable (not illustrated in FIG. 8) to the connector 805. According to this configuration, the biological information measurement device 108 and the patient terminal 106 are communicably connected. Specifically, the connector 805 functions as a connection interface 906 described later.

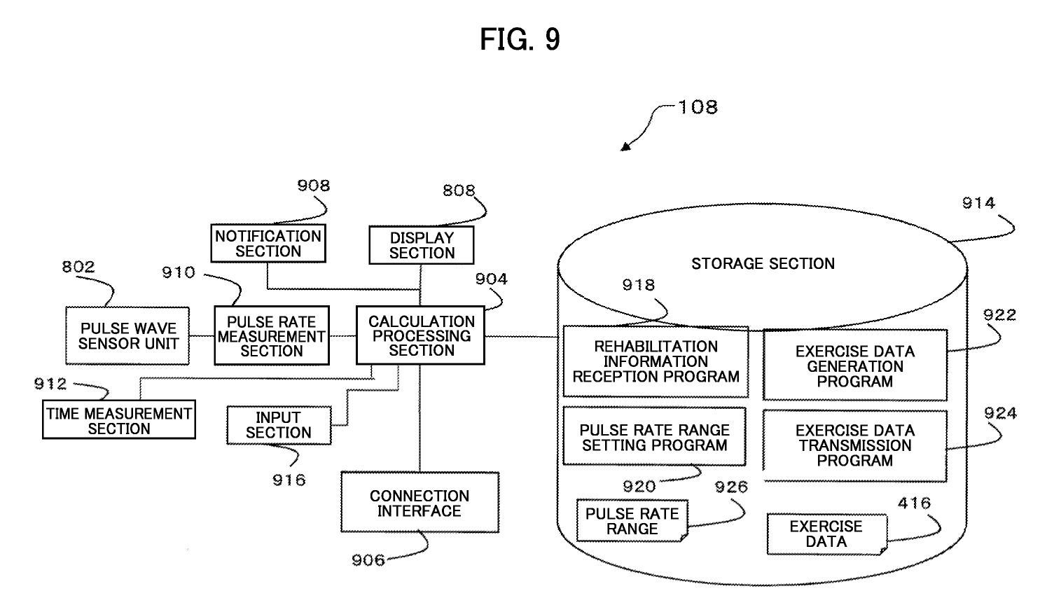

FIG. 9 is a block diagram illustrating the functional configuration of the biological information measurement device 108.

A storage section 914 stores a control program executed by a calculation processing section 904, and data. Examples of the data stored in the storage section 914 include a pulse rate range 926 and the exercise data 416, the pulse rate range 926 representing the upper limit and the lower limit of the pulse rate that are suitable when the patient performs rehabilitation exercise. Examples of the program stored in the storage section 914 include a rehabilitation information reception program 918 for receiving the rehabilitation information 300 from the patient terminal 106, a pulse rate range setting program 920 that sets the pulse rate range included in the rehabilitation information 300, an exercise data generation program 922 that generates the exercise data 416, and an exercise data transmission program 924 for transmitting the exercise data 416 to the patient terminal 106.

The calculation processing section 904 controls each section included in the biological information measurement device 108, and performs a calculation process. The calculation processing section 904 is a CPU, for example.

The calculation processing section 904 reads the rehabilitation information reception program 918 from the storage section 914, and executes the rehabilitation information reception program 918 to acquire the rehabilitation information 300 from the patient terminal 106 through the communication section 906. Specifically, the calculation processing section 904 functions as a rehabilitation information reception section.

The calculation processing section 904 reads the pulse rate range setting program 920 from the storage section 914, and executes the pulse rate range setting program 920 to set the pulse rate range 926 to the biological information measurement device 108 using the rehabilitation information 300. Specifically, the calculation processing section 904 functions as a pulse rate range setting section.

The calculation processing section 904 reads the exercise data generation program 922 from the storage section 914, and executes the exercise data generation program 922 to generate the exercise data 416. Specifically, the calculation processing section 904 functions as an exercise data generation section.

The calculation processing section 904 reads the exercise data transmission program 924 from the storage section 914, and executes the exercise data transmission program 924 to transmit the exercise data 416 to the patient terminal 106 through the connection interface 906. Specifically, the calculation processing section 904 functions as an exercise data transmission section (also referred to as “pulse measurement information transmission section”).

The connection interface 906 is an interface for connecting to the patient terminal 106. The connection interface 906 is implemented by a serial interface (e.g., Universal Serial Bus (USB) or IEEE1394), for example.

A time measurement section 912 measures time. The time measurement section 912 outputs the time measurement result to the calculation processing section 904. An input section 916 corresponds to the button switches 811 to 813 and the start/stop button 816. The input section 916 outputs a signal corresponding to a button operation performed by the patient to the calculation processing section 904. Note that the time measurement section 912 may measure the elapsed time from the pulse rate measurement start timing. In this case, the time measurement section 912 functions as an elapsed time measurement section.

A notification section 908 issues a notification to the patient using sound, vibrations, or the like. For example, the notification section 908 generates an alarm sound at a volume corresponding to the instruction from the calculation processing section 904. The notification section 908 may include a vibrating motor, and generates vibrations at a magnitude corresponding to the instruction from the calculation processing section 904. Note that the notification section 908 need not necessarily be provided.

The pulse wave sensor unit 802 detects pulse waves (i.e., the biological information about the patient), and outputs a pulse wave signal to a pulse rate measurement section 910. The configuration of the pulse wave sensor unit 802 is known in the art, and detailed description thereof is omitted. Note that it is preferable that the pulse wave sensor unit 802 include an LED and a phototransistor, and be configured so that light emitted from the LED is reflected by blood vessels situated under the skin of the patient, and received by the phototransistor, for example. The light received by the phototransistor is photoelectrically converted to obtain the pulse wave signal.

The pulse rate measurement section 910 calculates the pulse rate from the pulse wave signal output from the pulse wave sensor unit 802. The pulse rate measurement section 910 may have a known configuration. For example, the pulse rate measurement section 910 performs an amplification process, an analog/digital conversion process, and a fast Fourier transform (FFT) process on the pulse wave signal to calculate the frequency components of the pulse wave signal to calculate a pulse wave spectral signal.

A body motion component is removed from the calculated pulse wave spectral signal to calculate a pulse wave component. The body motion component can be calculated using an acceleration sensor, for example. The frequency fMmax of the pulse wave component is substituted into the expression (1) to calculate the pulse rate (beats/min).

Pulse rate (beats/min)=fMmax (Hz)×60 (1)

The pulse rate thus calculated is output to the calculation processing section 904. The pulse rate is measured at given intervals. For example, the pulse rate is measured at intervals of 5 seconds.

The display section 808 displays various types of information. The display section 808 displays information under control of the calculation processing section 904.

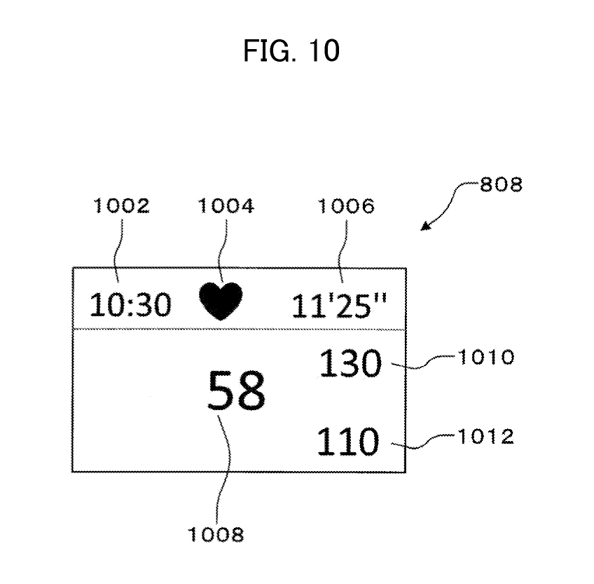

FIG. 10 is a view illustrating an example of the display state of the display section 808. A current time indicator 1002 displays the current time. In FIG. 10, the current time is “10:30”. A pulse measurement state indicator 1004 displays the pulse measurement state. For example, a heart mark blinks when the pulse rate is measured.

A pulse measurement time indicator 1006 displays the elapsed time from the timing at which pulse rate measurement has been started by pressing the start/stop button 816. In FIG. 10, 11 minutes and 25 seconds has elapsed from the timing at which pulse rate measurement has been started. Note that the pulse measurement time indicator 1006 may be omitted.

A pulse rate indicator 1008 displays the measured pulse rate. In FIG. 10, the pulse rate is “58” (beats/min). An upper-limit pulse rate indicator 1010 displays the upper-limit pulse rate of the pulse rate range 926 that has been set by the pulse rate range setting program 920. In FIG. 10, the upper-limit pulse rate is “130”. A lower-limit pulse rate indicator 1012 displays the lower-limit pulse rate of the pulse rate range 926 that has been set by the pulse rate range setting program 920. In FIG. 10, the lower-limit pulse rate is “110”. It is possible to allow the patient to be aware of the pulse rate range 926, and easily implement effective rehabilitation exercise by displaying the upper-limit pulse rate indicator 1010 and the lower-limit pulse rate indicator 1012 on the display section 808.

### 1-2. Process of Remote Rehabilitation Method

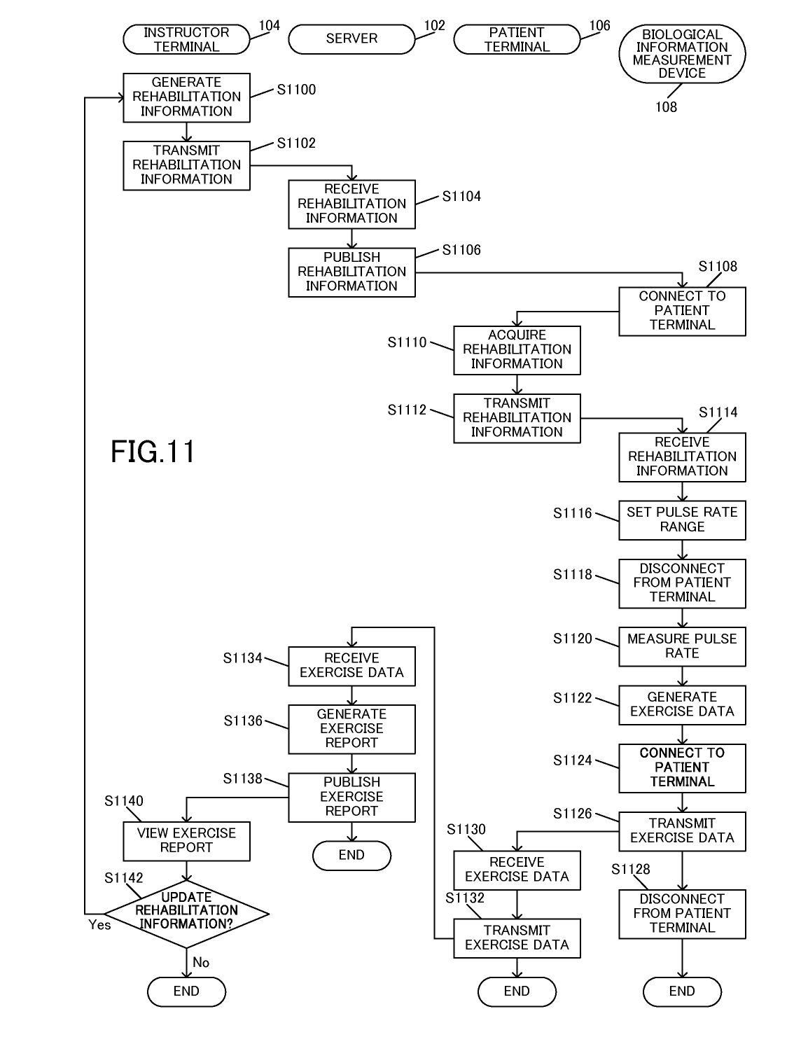

FIG. 11 is a flowchart illustrating an example of the process of the remote rehabilitation method that utilizes the remote rehabilitation system 100. The steps are sequentially described below in time series for convenience of explanation. Note that the steps need not necessarily be performed in the order described below. Some of the steps may be performed in reverse order, or may be performed in parallel.

The instructor generates the rehabilitation information 300 suitable for the patient using the input device 204 of the instructor terminal 104 (step S1100). Specifically, the instructor generates information about the pulse rate range that is suitable for the patient to exercise.

The instructor terminal 104 executes the rehabilitation information transmission program 212 to transmit the generated rehabilitation information 300 to the server 102 (step S1102).

The server 102 executes the rehabilitation information reception program 406. When the rehabilitation information reception program 406 has detected that the rehabilitation information 300 has been transmitted from the instructor terminal 104, the server 102 receives the rehabilitation information 300 (step S1104).

The server 102 executes the rehabilitation information publication program 408 to publish the received rehabilitation information 300 (step S1106). The server 102 may publish the received rehabilitation information 300 so that the rehabilitation information 300 can be acquired from the patient terminal 106 used by the patient represented by the patient ID 302 included in the rehabilitation information 300, and cannot be acquired from other patient terminals.

When the biological information measurement device 108 is connected to the patient terminal 106 through the connection interface 906 and the connection interface 712 (step S1108), the patient terminal 106 executes the rehabilitation information acquisition program 714 to acquire the rehabilitation information 300 published by the server 102 (step S1110).

The patient terminal 106 executes the rehabilitation information transmission program 716 to transmit the acquired rehabilitation information 300 to the biological information measurement device 108 through the connection interface 906 and the connection interface 712 (step S1112).

The biological information measurement device 108 executes the rehabilitation information reception program 918 to receive the rehabilitation information 300 from the patient terminal 106 (step S1114). The biological information measurement device 108 executes the pulse rate range setting program 920 to set the upper-limit pulse rate 306 and the lower-limit pulse rate 304 included in the received rehabilitation information 300 to be the pulse rate range 926 (step S1116). The biological information measurement device 108 is then disconnected from the patient terminal 106 (step S1118).

The patient wears the biological information measurement device 108 on the wrist, and performs rehabilitation exercise. Examples of exercise suitable for rehabilitation exercise include walking. When the patient has pressed the start/stop button 816 of the biological information measurement device 108, the pulse wave sensor unit 802 and the pulse rate measurement section 910 operate to start pulse rate measurement (step S1120). The time measurement section 912 may measure the elapsed time from the pulse rate measurement start timing. The patient exercises for a given time (e.g., 30 minutes).

The display section 808 displays the indicators illustrated in FIG. 10 during pulse rate measurement. The display section 808 displays the current pulse rate, and the upper-limit pulse rate and the lower-limit pulse rate of the pulse rate range 926. The biological information measurement device 108 executes the exercise data generation program 922 to generate the exercise data 416 in which the measured pulse rate and the measurement time are linked, and stores the generated exercise data 416 in the storage section 914 (step S1122). The exercise data 416 is stored in the storage section 914 as data in which the measurement time and the pulse rate are linked at intervals of 5 seconds, for example. The storage section 914 can store the exercise data 416 during a one-week period, for example.

When the given time has elapsed from the pulse rate measurement start timing, the notification section 908 notifies the patient that the given time has elapsed by generating sound or vibrations, and the patient stops exercise. The biological information measurement device 108 stops pulse rate measurement. Note that the notification section 908 need not necessarily notify the patient that the given time has elapsed. It is preferable that the notification section 908 notify the patient that the given time has elapsed. The patient then removes the biological information measurement device 108 from the wrist.

The patient performs rehabilitation exercise (for the given time) for an arbitrary period (e.g., 1 week), and connects the biological information measurement device 108 to the patient terminal 106 (step S1124). When the biological information measurement device 108 has detected that the biological information measurement device 108 has been connected to the patient terminal 106, the biological information measurement device 108 executes the exercise data transmission program 924 to transmit the exercise data 416 (e.g., during a one-week period) stored in the storage section 914 to the patient terminal 106 (step S1126). After transmitting the exercise data 416, the biological information measurement device 108 disconnects from the patient terminal 106 (step S1128).

When the patient terminal 106 has detected that the biological information measurement device 108 has been connected to the patient terminal 106, the patient terminal 106 executes the exercise data reception program 718 to receive the exercise data 416 from the biological information measurement device 108 (step S1130). The patient terminal 106 executes the exercise data transmission program 720 to transmit the exercise data 416 to the server 102 (step S1132).

The server 102 executes the exercise data reception program 410. When the exercise data reception program 410 has detected that the exercise data 416 has been transmitted from the patient terminal 106, the server 102 receives the exercise data 416 (step S1134).

The server 102 executes the exercise report generation program 412 once a week to generate the exercise report 418 illustrated in FIG. 6 (step S1136). The server 102 executes the exercise report publication program 414 to publish the generated exercise report 418 (step S1138). It is preferable that the server 102 publish the exercise report 418 so that the exercise report 418 can be viewed from the instructor terminal 104, and cannot be viewed from other terminals.

The instructor views the exercise report 418 of each patient using the instructor terminal 104 (step S1140). The instructor checks the exercise report 418 of each patient, and determines whether or not to update the rehabilitation information 300 about each patient (step S1142). When the instructor has determined that it is unnecessary to update the rehabilitation information 300 (step S1142: No), the instructor allows the rehabilitation information 300 to remain unchanged. When the instructor has determined that it is necessary to update the rehabilitation information 300 (step S1142: Yes), the instructor generates the rehabilitation information 300 in the step S1100. Specifically, when the health condition of the patient has been improved by rehabilitation exercise, the instructor increases or decreases the pulse rate range suitable for the patient to exercise.

### 1-3. Advantageous Effects

According to the first embodiment, the instructor terminal 104 can transmit the rehabilitation information 300 including the information about the pulse rate range applied to the patient to the server 102 to set the pulse rate range applied to the biological information measurement device 108 through the server 102 and the patient terminal 106. This makes it unnecessary for the patient to set the pulse rate range, and makes it possible to improve convenience to the patient. Since the instructor (e.g., doctor) can remotely set the pulse rate range applied to the patient, the instructor can set the pulse rate range suitable for the patient at an appropriate timing corresponding to an improvement in the health condition of the patient through rehabilitation. Therefore, the effects of remote rehabilitation can be further improved.

The instructor terminal 104 transmits the rehabilitation information 300 including the information about the pulse rate range instead of transmitting an exercise prescription program. This makes it possible to implement rehabilitation exercise corresponding to the physical condition of the patient. When the physical condition of the patient is bad, the pulse rate of the patient normally easily reaches the set pulse rate range even when the patient performs relatively mild exercise. When the physical condition of the patient is good, the pulse rate of the patient does not reach the set pulse rate range unless the patient performs relatively hard exercise. Specifically, it is possible to implement rehabilitation exercise corresponding to the physical condition of the patient by setting the pulse rate range.

The server 102 receives the exercise data 416 including the pulse rate measured by the biological information measurement device 108 and the measurement time at which the pulse rate was measured, through the patient terminal 106, generates the exercise report 418 based on the received exercise data 416, and allows the generated exercise report 418 to be viewed from the instructor terminal 104. This makes it possible to report the status of implementation of rehabilitation exercise performed by the patient to the instructor in a comprehensible manner, and the instructor can determine an improvement in the health condition of the patient through rehabilitation exercise, and set the pulse rate range suitable for the patient at an appropriate timing.

The biological information measurement device 108 displays the set pulse rate range. This makes it possible to allow the patient to be aware of the pulse rate range set for the patient, and easily implement effective rehabilitation exercise. It is possible to allow the patient to be aware of the pulse rate range set for the patient by displaying the pulse rate range set for the patient based on a novel finding that the time in which the patient exercised within the pulse rate range set by the instructor (e.g., doctor) improves the effects of rehabilitation exercise performed by the patient.

### 2. Second Embodiment

The invention is not limited to the first embodiment. The invention can be implemented in various ways without departing from the scope of the invention. The second embodiment, the third embodiment, and the modifications are described below. Note that the same elements as those described above in connection with the first embodiment are represented by the same reference signs, and description thereof is omitted.

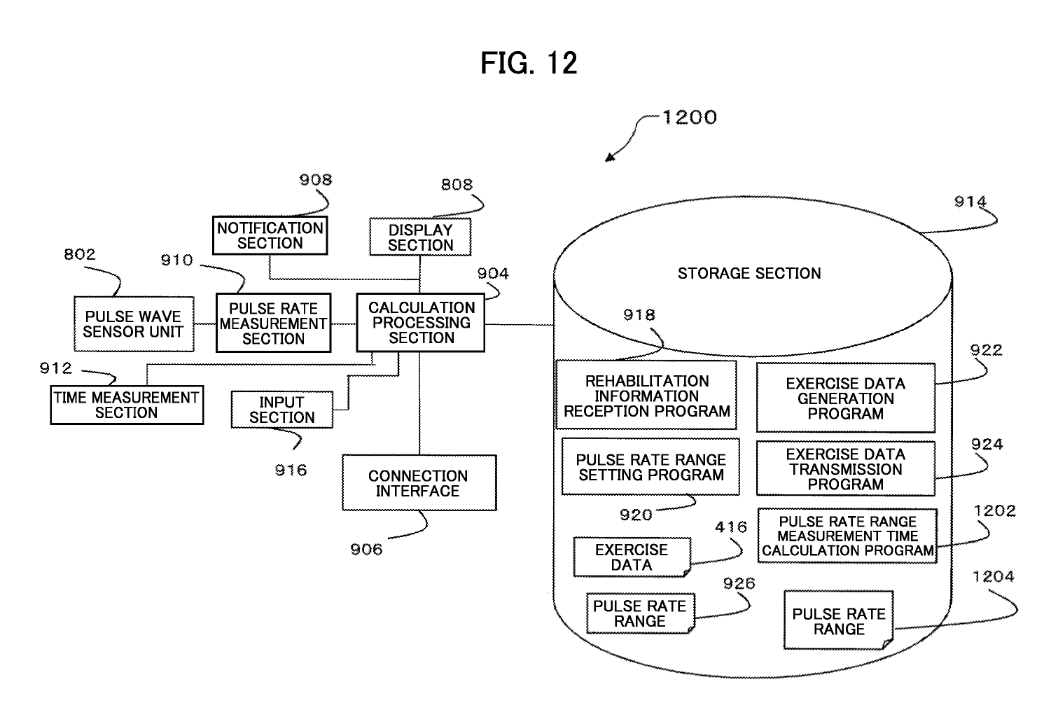

FIG. 12 is a functional block diagram of a biological information measurement device 1200 according to the second embodiment. The biological information measurement device 1200 differs from the biological information measurement device 108 according to the first embodiment in that a pulse rate range measurement time calculation program 1202 is stored in the storage section 914.

The calculation processing section 904 reads the pulse rate range measurement time calculation program 1202 from the storage section 914, and executes the pulse rate range measurement time calculation program 1202 to calculate the cumulative time (pulse rate range measurement time) in which the pulse rate measured by the pulse rate measurement section 910 fell within the pulse rate range 926. Specifically, the calculation processing section 904 functions as a pulse rate range measurement time calculation section.

The cumulative time may be calculated by determining whether or not two pieces of pulse rate data 504 that are adjacent to each other in time series (i.e., two pieces of corresponding measurement date/time data 502 are adjacent to each other in time series) are within the pulse rate range 926 referring to the exercise data 416, and adding the time in which the two pieces of pulse rate data 504 are within the pulse rate range 926 to the cumulative time, for example.

The calculation processing section 904 stores the cumulative time calculated by the pulse rate range measurement time calculation program 1202 in the storage section 914 as a pulse rate range measurement time 1204, and displays the cumulative time on the display section 808.

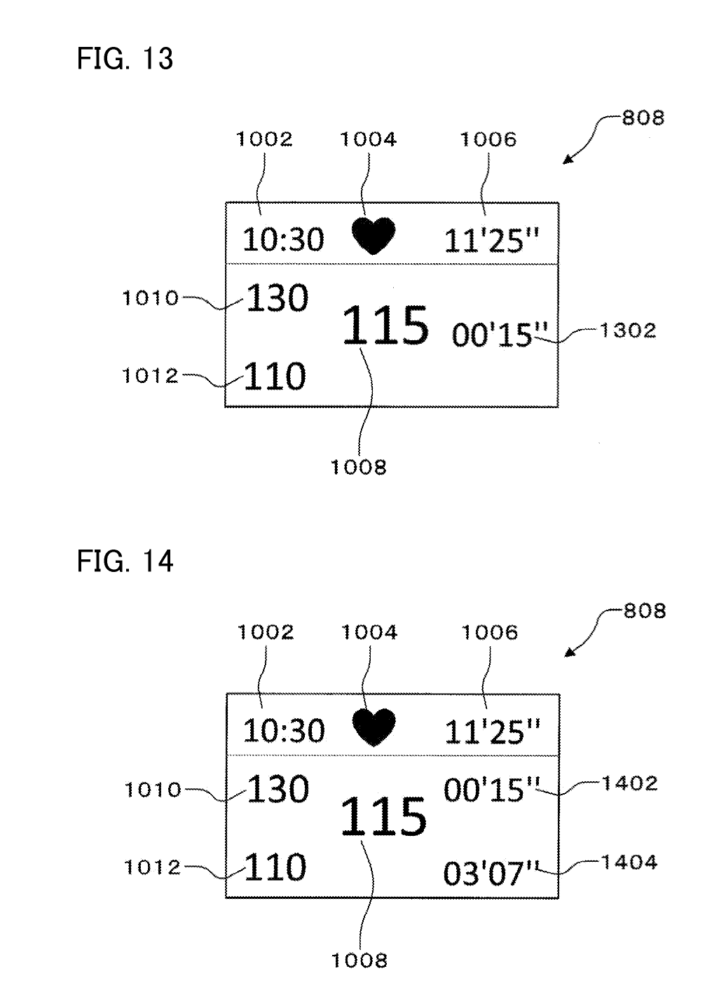

FIG. 13 is a view illustrating an example of the display state of the display section 808 according to the second embodiment. The display state illustrated in FIG. 13 differs from the display state illustrated in FIG. 10 in that the display position of the upper-limit pulse rate indicator 1010 and the display position of the lower-limit pulse rate indicator 1012 are changed as compared with FIG. 10, and a pulse rate range measurement time indicator 1302 that displays the pulse rate range measurement time 1204 is additionally provided. FIG. 13 illustrates an example in which the cumulative time in which the measured pulse rate was within the pulse rate range “110 to 130” was 15 seconds.

It is possible to allow the patient to be aware of the pulse rate range measurement time, and make an effort to increase to the pulse rate range measurement time 1204 by displaying the pulse rate range measurement time indicator 1302 on the display section 808. This makes it possible to allow the patient to determine whether or not the patient has performed effective rehabilitation exercise.

The notification section 908 may issue a notification to the patient as described below along with implementation of the configuration in which the pulse rate range measurement time 1204 is calculated by the pulse rate range measurement time calculation program 1202.

In the first embodiment, when a given time (e.g., 30 minutes) has elapsed from the pulse rate measurement start timing, the notification section 908 notifies the patient that the given time has elapsed by generating sound or vibrations, and the patient stops exercise in response to the notification. However, the patient may stop exercise before the given time elapses as long as the patient exercises within the pulse rate range 926 for a sufficient time.

Therefore, when the pulse rate range measurement time 1204 has reached a first time (e.g., 15 minutes) that is shorter than the given time, the notification section 908 may notify the patient to that effect. This makes it possible to notify the patient that the patient has performed sufficient rehabilitation exercise before the given time elapses. This makes it possible to implement more effective rehabilitation exercise.

When the pulse rate range measurement time 1204 has not reached the first time until the given time elapses, the notification section 908 may notify the patient that the given time (also referred to as “second time”) has elapsed in the same manner as in the first embodiment. In this case, it is desirable to issue the notification in a way differing from that employed when notifying the patient that the pulse rate range measurement time 1204 has reached the first time. For example, it is desirable to notify the patient that the pulse rate range measurement time 1204 has reached the first time in such a way that the patient is positively impressed that the patient could perform sufficient rehabilitation exercise. It is desirable to notify the patient that the second time has elapsed from the pulse rate measurement start timing in such a way that the patient is negatively impressed that the patient could not perform sufficient exercise within the pulse rate range 926 although the patient performed exercise for a sufficient time.

This makes it possible to notify the patient that the patient can stop exercise although the pulse rate range measurement time 1204 has not reached the first time. This makes it possible to prevent a situation in which excessive continuous exercise threatens the life of the patient, and implement safe rehabilitation exercise.

The first time or the second time may be a predetermined time, or may be changed corresponding to the patient. When changing the first time or the second time corresponding to the patient, information about the first time or the second time may be incorporated in the rehabilitation information 300, and the instructor may remotely set the first time or the second time to the biological information measurement device 1200 corresponding to the patient together with the pulse rate range 926.

### 3. Third Embodiment

FIG. 14 is a view illustrating an example of the display state of the display section 808 according to the third embodiment. The display state illustrated in FIG. 14 differs from the display state illustrated in FIG. 13 in that an upper-half measurement time indicator 1402 and a lower-half measurement time indicator 1404 are displayed instead of the pulse rate range measurement time indicator 1302.

The upper-half measurement time indicator 1402 displays the cumulative measurement time (upper range measurement time) in which the pulse rate measured by the pulse rate measurement section 910 was within a given range from the upper limit of the pulse rate range 926. It is preferable that the given range be a range from the upper limit of the pulse rate range 926 to a value calculated by adding 1 to the median value of the pulse rate range 926. FIG. 14 illustrates an example in which the cumulative time in which the pulse rate was within the range “121 to 130” was 15 seconds.

The lower-half measurement time indicator 1404 displays the cumulative measurement time in which the pulse rate measured by the pulse rate measurement section 910 was within a given range from the lower limit of the pulse rate range 926. It is preferable that the given range be a range from the lower limit of the pulse rate range 926 to the median value of the pulse rate range 926. FIG. 14 illustrates an example in which the cumulative time in which the pulse rate was within the range “110 to 120” was 3 minutes and 7 seconds. Note that the lower-half measurement time indicator 1404 may be omitted.

When implementing the display state illustrated in FIG. 14, the pulse rate range measurement time calculation program 1202 calculates the cumulative time in which the pulse rate measured by the pulse rate measurement section 910 was within the upper-half range (i.e., the range from the upper limit to the median value) of the pulse rate range 926, and the cumulative time in which the pulse rate measured by the pulse rate measurement section 910 was within the lower-half range (i.e., the range from the lower limit to the median value) of the pulse rate range 926.

The inventors found as a result of extensive studies that the effects of rehabilitation exercise performed by the patient are improved when the patient exercises for a long time at a pulse rate within a range close to the upper limit of the pulse rate range. Therefore, it is possible to allow the patient to determine whether or not the patient could perform effective rehabilitation exercise by displaying the upper range measurement time that is the cumulative time in which the pulse rate was within a given range from the upper limit of the pulse rate range.

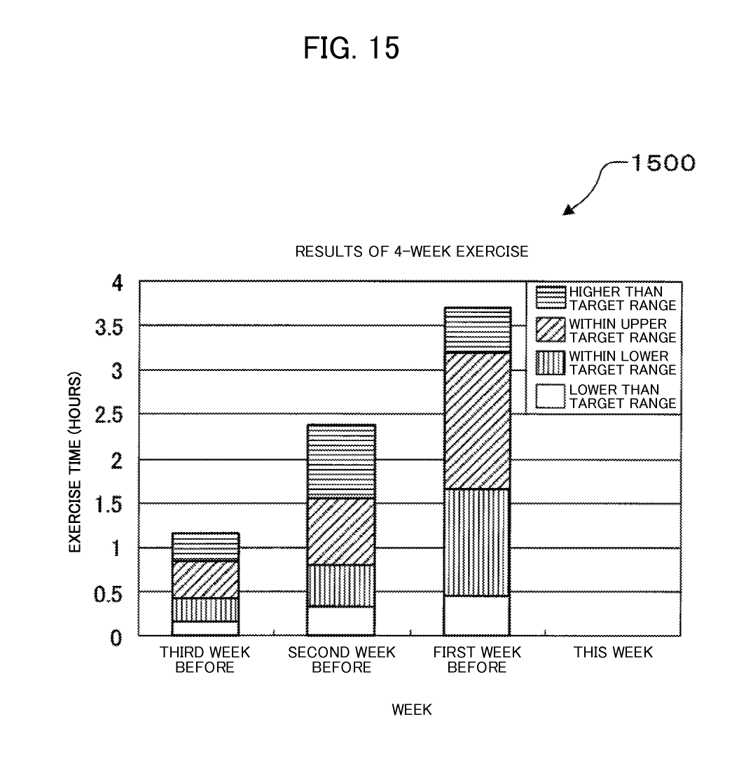

The server 102 may generate the exercise report illustrated in FIG. 15 based on the finding that the effects of rehabilitation exercise performed by the patient are improved when the patient exercises for a long time at a pulse rate within a range close to the upper limit of the pulse rate range.

FIG. 15 illustrates an example of an exercise report 1500 generated by the server 102 according to the third embodiment. The exercise report 1500 illustrated in FIG. 15 differs from the exercise report 418 illustrated in FIG. 6 as to the details of each vertical bar graph.

In FIG. 15, the vertically shaded part of each vertical bar graph represents the cumulative time in which the patient exercised at a pulse rate within the lower-half range (i.e., the range from the lower limit to the median value) of the pulse rate range (target) suitable for the patient. The diagonally shaded part of each vertical bar graph represents the cumulative time in which the patient exercised at a pulse rate within the upper-half range (i.e., the range from the median value to the upper limit) of the pulse rate range suitable for the patient.

This makes it possible to allow the instructor to more easily determine whether or not the patient could perform effective rehabilitation exercise when checking the exercise report 1500.

When the server 102 generates the exercise report 1500 illustrated in FIG. 15, the exercise report generation program 412 must calculate the cumulative time in which the patient exercised at a pulse rate within the lower-half range (i.e., the range from the lower limit to the median value) of the pulse rate range (target) suitable for the patient, and the cumulative time in which the patient exercised at a pulse rate within the upper-half range (i.e., the range from the median value to the upper limit) of the pulse rate range suitable for the patient, referring to the rehabilitation information 300 and the exercise data 416.

### 4. Modifications

The invention is not limited to the above embodiments. The invention can be implemented in various ways without departing from the scope of the invention. For example, it is possible to implement the following modifications.

### 4-1. Display State of Biological Information Measurement Device

The display screen displayed on the biological information measurement device is not limited to those described in connection with the above embodiments. For example, the following configuration may also be employed.

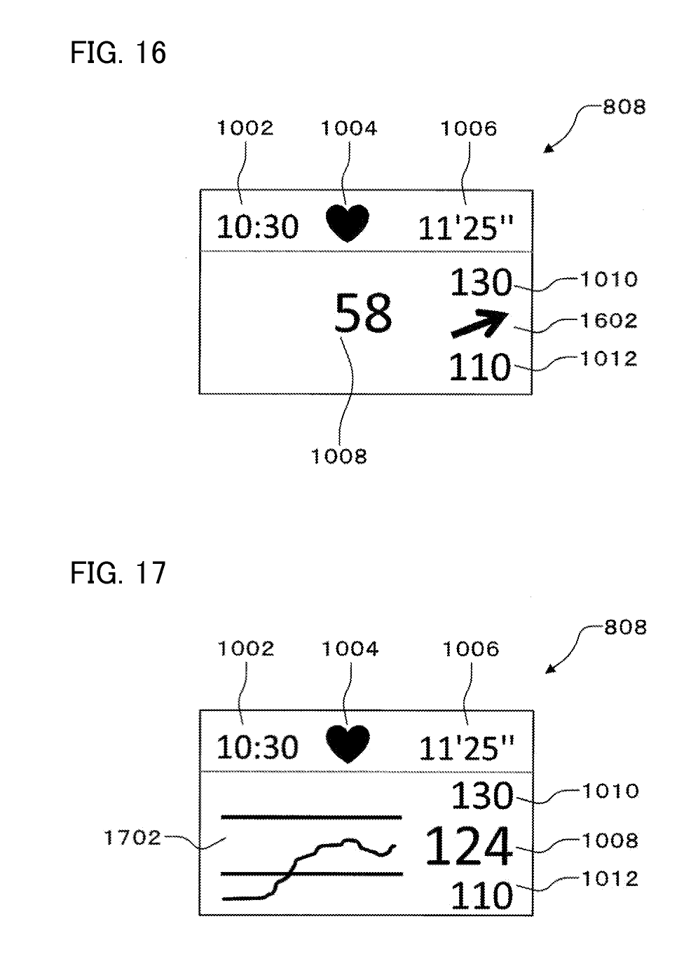

FIG. 16 is a view illustrating an example of the display screen displayed on the biological information measurement device. The display screen illustrated in FIG. 16 is characterized in that a patient instruction indicator 1602 is displayed. The patient instruction indicator 1602 is an indicator that instructs the patient to increase the pulse rate using an upper-right-pointing arrow, or instructs the patient to decrease the pulse rate using a lower-right-pointing arrow, or instructs the patient to maintain the current pulse rate using a right-pointing arrow. The patient can easily determine whether he should perform harder exercise (i.e., increase the pulse rate), or should perform milder exercise (i.e., decrease the pulse rate) since the intensity of exercise is too high, or should perform exercise at the same intensity (i.e., maintain the pulse rate) by displaying the patient instruction indicator 1602.

FIG. 17 is a view illustrating another example of the display screen displayed on the biological information measurement device. The display screen illustrated in FIG. 17 is characterized in that a pulse rate change indicator 1702 is displayed. The pulse rate change indicator 1702 displays a change in pulse rate from the pulse rate measurement start timing. The patient can determine the pulse rate change tendency, and adjust the intensity of exercise corresponding to the pulse rate change tendency by displaying the pulse rate change indicator 1702. The pulse rate change display period may be narrowed when a change in the pulse rate of the patient is not stable, or when the amount of data increases due to an increase in measurement time. For example, the patient can easily determine the current pulse rate change tendency when the pulse rate change display period is set to 2 minutes.

### 4-2. Configuration of Biological Information Measurement Device

The above embodiments have been described taking a wristwatch-type device that measures the pulse waves from the finger of the patient as an example of the biological information measurement device. Note that the biological information measurement device may be a wristwatch-type device that measures the pulse waves from the wrist of the patient, or may be a belt-type device that is wound around the chest of the patient, and measures the heart rate.

Although FIG. 8 illustrates an example in which the connector 805 is provided to the periphery of the main body 800 at a position corresponding to the six o'clock direction, and the connector piece 806 is removably attached, the configuration is not limited thereto. For example, a removable mechanism such as the connector 805 and the connector piece 806 may not be provided, and a band member used for a wristwatch may be attached to the main body 800 so that the main body 800 can be removably attached to the body of the patient, or an elastic material may be attached to the main body 800 so that the main body 800 can be removably attached to the body of the patient.

### 4-3. Type of Rehabilitation Exercise

The above embodiments have been described taking walking as an example of rehabilitation exercise. Note that rehabilitation exercise is not limited thereto. Various types of rehabilitation exercise such as climbing, running, gymnastic exercise, yoga, swimming, or walking in water may also be used. When rehabilitation exercise is walking in water, it is preferable that the biological information measurement device have sufficient waterproofness.

### 4-4. Type of Disease

The above embodiments have been described taking a patient with heart disease as an example of the patient. Note that the patient is not limited thereto. For example, the above embodiments may also be applied to a patient with brain infarction or peripheral artery disease. Since exercise rehabilitation aims to improve the blood flow of the patient, the above embodiments may also be applied to a patient with atherothrombosis who shows a deterioration in vascular function (e.g., vascular endothelial dysfunction or vasoconstriction) due to arteriosclerosis. The above embodiments may also be applied to a patient with metabolic syndrome who may develop atherothrombosis, or a patient with lifestyle-related disease such as diabetes, hypertension, or hyperlipidemia.

### 4-5. Communications Between Patient and Instructor

The above embodiments have been described taking an example in which the patient and the instructor rarely communicate with each other during remote rehabilitation. Note that the system may be designed so that the patient and the instructor communicate with each other using the patient terminal and the instructor terminal. For example, the system may be designed so that the patient and the instructor communicate with each other through an Internet phone since the patient terminal and the instructor terminal are connected to the network. In this case, since the patient can receive instructions directly from the instructor during remote rehabilitation, it is possible to alleviate a patient's anxiety.

### 4-6. Patient Terminal

Although the above embodiments have been described taking an example in which the patient terminal 106 illustrated in FIG. 7 is a personal computer, the configuration is not limited thereto. For example, the patient terminal 106 may be an information terminal device having an external communication function, such as a smartphone.

Although the above embodiments have been described taking an example in which the patient terminal 106 illustrated in FIG. 7 includes the rehabilitation information transmission program 716 for transmitting the rehabilitation information 300 to the biological information measurement device 108, the configuration is not limited thereto. The patient terminal 106 may not include the rehabilitation information transmission program 716, and may be configured to transmit the rehabilitation information 300 acquired from the server 102 directly to the biological information measurement device 108.

Although only some embodiments of the present invention have been described in detail above, those skilled in the art will readily appreciate that many modifications are possible in the embodiments without materially departing from the novel teachings and advantages of this invention. Accordingly, all such modifications are intended to be included within scope of this invention.

What is claimed is:

1. A biological information measurement device comprising:
a rehabilitation information reception section that receives rehabilitation information that includes information about a pulse rate range applied to a patient;
a pulse rate range setting section that sets the pulse rate range based on the rehabilitation information;
a display section that displays at least the pulse rate range;
a pulse rate measurement section that measures a pulse rate of the patient;
a storage section that stores pulse measurement information that includes the pulse rate measured by the pulse rate measurement section, and a measurement time at which the pulse rate was measured; and
a pulse measurement information transmission section that transmits the pulse measurement information.

2. The biological information measurement device as defined in claim 1, further comprising:
a pulse rate range measurement time calculation section that calculates a pulse rate range measurement time in which the measured pulse rate was within the pulse rate range,
the display section displaying the pulse rate range measurement time.

3. The biological information measurement device as defined in claim 2,
the pulse rate range measurement time calculation section calculating an upper range measurement time in which the measured pulse rate was within a given range from an upper limit of the pulse rate range, and
the display section displaying at least the upper range measurement time.

4. The biological information measurement device as defined in claim 2, further comprising:
a notification section that issues a notification to the patient when the pulse rate range measurement time has reached a first time.

5. The biological information measurement device as defined in claim 3, further comprising:
a notification section that issues a notification to the patient when the pulse rate range measurement time has reached a first time.

6. The biological information measurement device as defined in claim 4, further comprising:
an elapsed time measurement section that measures an elapsed time from start of the measurement of the pulse rate,
the notification section issuing a notification to the patient when the elapsed time has reached a second time that is longer than the first time.

7. The biological information measurement device as defined in claim 5, further comprising:
an elapsed time measurement section that measures an elapsed time from start of the measurement of the pulse rate,
the notification section issuing a notification to the patient when the elapsed time has reached a second time that is longer than the first time.

8. A patient terminal comprising:
a rehabilitation information acquisition section that acquires rehabilitation information from a server, the rehabilitation information including information about a pulse rate range applied to a patient;
a rehabilitation information transmission section that transmits the rehabilitation information to a biological information measurement device;
a pulse measurement information reception section that receives pulse measurement information from the biological information measurement device, the pulse measurement information including a measured pulse rate of the patient, and a measurement time at which the pulse rate was measured; and
a pulse measurement information transmission section that transmits the pulse measurement information to the server.

9. A server comprising:
a rehabilitation information reception section that receives rehabilitation information from an instructor terminal, the rehabilitation information including information about a pulse rate range applied to a patient;
a rehabilitation information publication section that allows the rehabilitation information to be acquired from a patient terminal;
a pulse measurement information reception section that receives pulse measurement information from the patient terminal, the pulse measurement information including a measured pulse rate of the patient, and a measurement time at which the pulse rate was measured;
a document information generation section that generates document information based on the pulse measurement information, the document information being information about a pulse rate range measurement time in which the measured pulse rate was within the pulse rate range; and
a document information publication section that allows the document information to be viewed from the instructor terminal.

10. A remote rehabilitation method that utilizes a server, an instructor terminal, a patient terminal, and a biological information measurement device, the remote rehabilitation method comprising:
causing the instructor terminal to transmit rehabilitation information to the server, the rehabilitation information including information about a pulse rate range applied to a patient;
causing the server to receive the rehabilitation information;
causing the server to allow the rehabilitation information to be acquired from the patient terminal;
causing the patient terminal to acquire the rehabilitation information from the server;
causing the patient terminal to transmit the rehabilitation information to the biological information measurement device;
causing the biological information measurement device to receive the rehabilitation information;
causing the biological information measurement device to set the pulse rate range based on the rehabilitation information;
causing the biological information measurement device to measure a pulse rate of the patient;
causing the biological information measurement device to store pulse measurement information that includes the measured pulse rate, and a measurement time at which the pulse rate was measured;
causing the biological information measurement device to transmit the pulse measurement information to the patient terminal;
causing the patient terminal to receive the pulse measurement information;
causing the patient terminal to transmit the pulse measurement information to the server;
causing the server to receive the pulse measurement information;
causing the server to generate document information based on the pulse measurement information, the document information being information about a pulse rate range measurement time in which the measured pulse rate was within the pulse rate range; and
causing the server to allow the document information to be viewed from the instructor terminal.
